(19) United States
(12) Patent Application Publication
(10) Pub. No.: US 2010/0179448 A1
(43) Pub. Date: Jul. 15, 2010

(54) Device and Method for Measuring a Physical Parameter in an Anatomic Organ

(75) Inventors: Svante Höjer, Kungalv (SE); Sten Holm, Molnlycke (SE); Ulf Johansson, Onsala (SE)

Correspondence Address: SCHWEGMAN, LUNDBERG & WOESSNER, P.A., P.O. BOX 2938, MINNEAPOLIS, MN, 55402, US

(73) Assignee: Samba Sensors AB, Västra Frölunda (SE)

(21) Appl. No.: 12/085,207

(22) PCT Filed: Nov. 21, 2006
(86) PCT No.: PCT/SE2006/050497

§ 371 Date: Dec. 28, 2009

(30) Foreign Application Priority Data
Nov. 21, 2005 (SE) 0502535-8

(51) Int. Cl. A61B 5/03

(52) U.S. Cl. 600/561

(57) ABSTRACT

The invention relates to a device for measuring a physical magnitude (p) in an anatomic organ (2) in human beings and animals, comprising a tubular, hollow cannula (8) arranged to be inserted into said human being or animal and comprising an end part (10) that ends in said organ (2), and an optical fibre (13) that is adapted to be arranged with an extension inside said cannula (8). The invention comprises the sensor device (18) that is arranged on said optical fibre (13) and which during use is positioned in connection to said organ (2) and is arranged for measuring said magnitude (p). The invention also relates to a method for such a measurement.

### TECHNICAL FIELD

The present invention relates to a device for measuring a physical magnitude in an anatomic organ in human beings and animals, comprising a tubular, hollow cannula arranged to be inserted into said human being or animal and comprising an end part that ends into said organ, and an optical fibre adapted to be arranged with an extension inside said cannula.

The invention also relates to a method for measuring a physical magnitude in an anatomic organ of human beings and animals, comprising the insertion of a tubular, hollow cannula into said human being or animal, where the end part of the cannula is positioned in said organ, and the insertion of an optical fibre with an extension inside said cannula.

### BACKGROUND ART

At certain states of illness that effect human vertebrae, for example when a disc has slipped, at so-called whiplash injuries or other back problems, there is a need of instruments and measuring methods for detection and analysis of the condition of the core of the respective disc in the vertebras. The core of the respective disc, called nucleus pulposus, constitutes a central part of the disc and comprises a semi-liquid, gelatinous substance. The nucleus pulposus is surrounded by a peripheral part, called annulus fibrosus, which consists of thread cartilage.

From the patent document U.S. Pat. No. 5,865,833 it is previously known to use a catheter which is shaped as a needle which point is inserted into nucleus pulposus with the intention to treat tissue by means of laser light.

Further, by the document US 2004/0127893 a cannula is previously known through which an optical fibre may be guided, with a purpose to admit visual study of the tissue in nucleus pulposus.

In the light of the above, it may be established that there is a need of devices and methods which are based on optical fibre technology and which are intended for measuring physical magnitudes, which in turn may constitute symptoms of states of illness of an anatomic organ in a human being or an animal, for example in nucleus pulposus.

### SUMMARY

A principal object of the present invention is thus to satisfy said needs and render a simple but still accurate measurement of a certain physical magnitude in an anatomic organ possible.

In particular, but not exclusively, the invention aims to admit a simple and accurate measurement of the pressure in nucleus pulposus.

The object above is achieved by means of a device as mentioned initially, which comprises a sensor device which is arranged on said optical fibre and which, during use, is positioned in connection with said organ and is arranged for measuring said magnitude.

The object is also achieved by means of a method as mentioned initially, which comprises positioning of a sensor device arranged on said optical fibre in connection with said organ, and measurement of said magnitude by means of said sensor device.

### BRIEF DESCRIPTION OF THE DRAWINGS

The invention will in the following be described in connection with preferred embodiment examples and the appended drawings, where

FIG. 1 is a schematic view, partly cross-sectional, of a device for measuring in accordance with the present invention, during use in connection with taking specimens in a human being's body;

FIG. 2 shows a perspective view of the invention, partly disassembled, and slightly enlarged in relation to FIG. 1;

FIG. 3 shows a perspective view of the invention, partly disassembled, and from another angle compared to FIG. 2;

FIG. 4 is a sectional view that shows a sensor device intended to be used at the invention;

FIG. 5 is a schematic cross-sectional view that shows a stopping device according to the invention; and

FIG. 6 is another schematic cross-sectional view that shows the function of the stopping device during use of the invention.

### PREFERRED EMBODIMENT

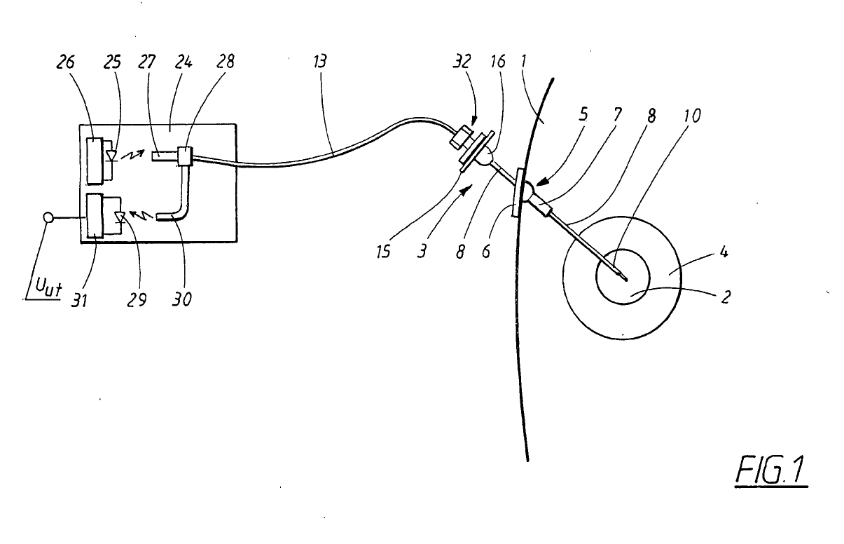

FIG. 1 schematically shows a device for measuring according to the invention, viewed in a cross-sectional view which also shows a part of a living creature's body 1. In the following, the invention will also be described with reference to a preferred embodiment example intended for measuring in a human body 1. The invention can, however, also be used in connection with a corresponding type of measurement in an animal's body.

More in detail, FIG. 1 shows the invention during use in connection with a measuring procedure for measurement of the pressure in nucleus pulposus 2, which as mentioned above constitutes the core of a spinal disc of the human body 1. The nucleus pulposus 2 comprises a semi-liquid, gelatinous substance. At certain states of illness, for example a slipped disc, it may be desired to measure a physical magnitude, such as the pressure p, inside the nucleus pulposus 2. By this reason, the invention is suitably arranged as a measuring device 3 for such a pressure measurement. As mentioned above, the nucleus pulposus 2 is surrounded by an annular part called the annulus fibrosus 4. The different parts comprised in the invention will in the following be described more in detail.

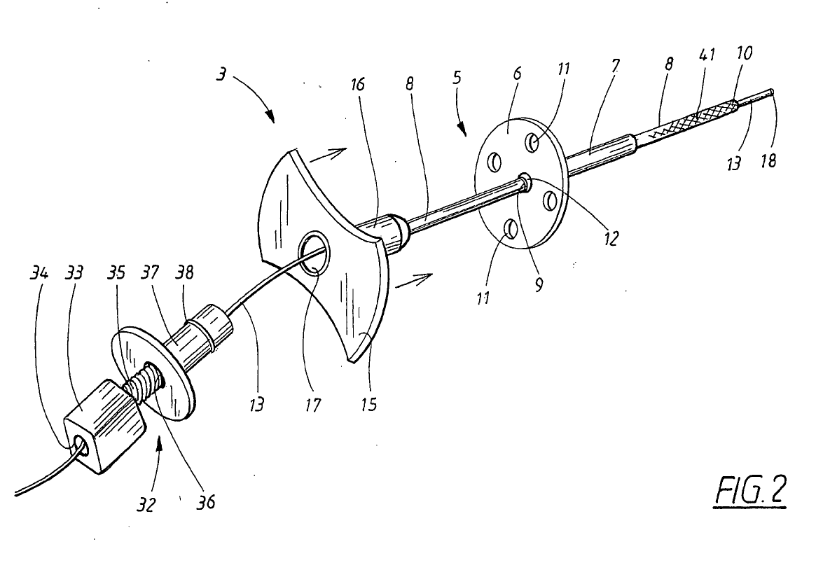

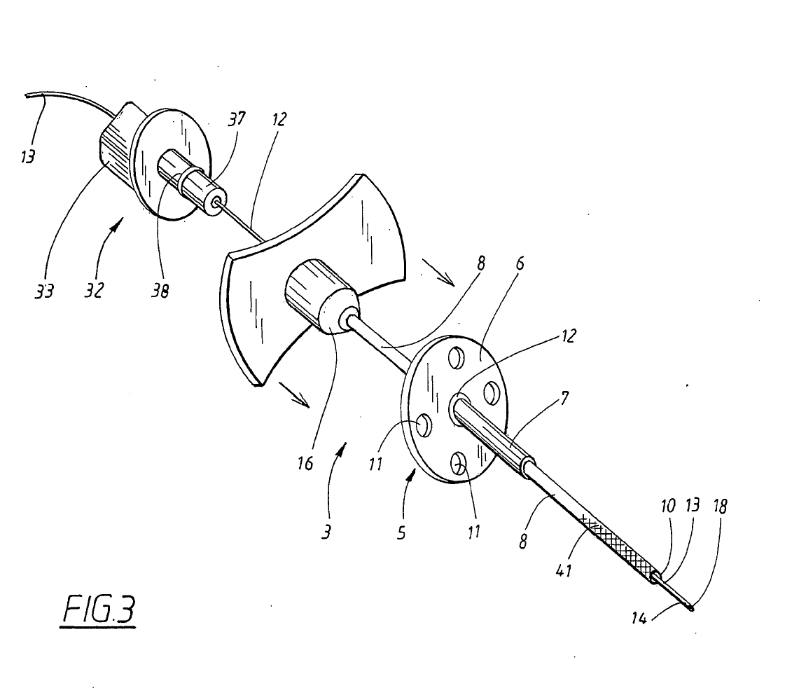

FIG. 1 shows the measuring device 3 according to the invention during use and in an assembled condition. FIG. 2 and FIG. 3, however, show the invention partly disassembled, and more in detail than FIG. 1. FIG. 2 and FIG. 3 show the invention in a perspective view from two different angles.

With reference to FIG. 1, FIG. 2 and FIG. 3, it is apparent that the measuring device 3 comprises a certain guide element 5, which preferably is shaped as a washer-like or flange-like element 6 in which an articulated tube 7 is arranged. The articulated tube 7 is arranged to be angled, i.e. to be positioned with different angular positions in relation to the flange element 6. Furthermore, the tube 7 is tubular, i.e. hollow. During measurement, the guide element 5 is arranged to bear on, and preferably also to be attached to, the human body's 1 skin. In connection with this, the articulated tube 7 shall be inserted in the body 1, according to what is shown in FIG. 1.

The guide element 5 is used to receive and guide a hollow cannula 8, i.e. a thin tube of a previously known kind. More in detail, this is performed by guiding the cannula 8 through a hole 9 which is defined in one of the ends of the articulated tube 7 (see FIG. 2). Furthermore, the cannula is brought all the way through the articulated tube 7 such that the cannula 8 protrudes out of the tube 7 for a certain distance. For this purpose, the inner diameter of the articulated tube 7 is slightly larger than the outer diameter of the cannula 8.

During use (see FIG. 1), the cannula 8 is intended to be inserted through the guide element 5 in such a way that the end part or point 10 of the cannula 8 is positioned inside nucleus pulposus 2. The tube 7 is then inserted through the skin and is mainly directed against the position for nucleus pulposus 2. The guide element 5 with the articulated tube 7 thus enables guidance of the cannula 8 from the position where the cannula 8 is inserted in the body 1. This enables the cannula 8 to be directed inwards towards nucleus pulposus 2 mainly independently of where the guide element 5 is placed and from which direction the cannula 8 is inserted.

According to what is apparent from in particular FIG. 2 and FIG. 3, the flange element 6 is provided with a number of openings 11. These openings 11 are intended to admit that the flange element 6, and thus the complete guide element 5, is sewed to, or in some other ways brought against, the skin of the human body 1 during use.

As also mentioned above, it is apparent from the FIGS. 2 and 3 that the conducting tube 7 is hollow in such a way that the cannula 8 may run inside the tube 7. Furthermore, the part of the tube 7 that is arranged at the flange element 6 is shaped like a ball joint 12, i.e. with a ball-like ending that is retained in a corresponding cavity in the flange element 6 and that admits that the tube 7 may be angled relatively freely in relation to the plane along which the flange element 6 is oriented.

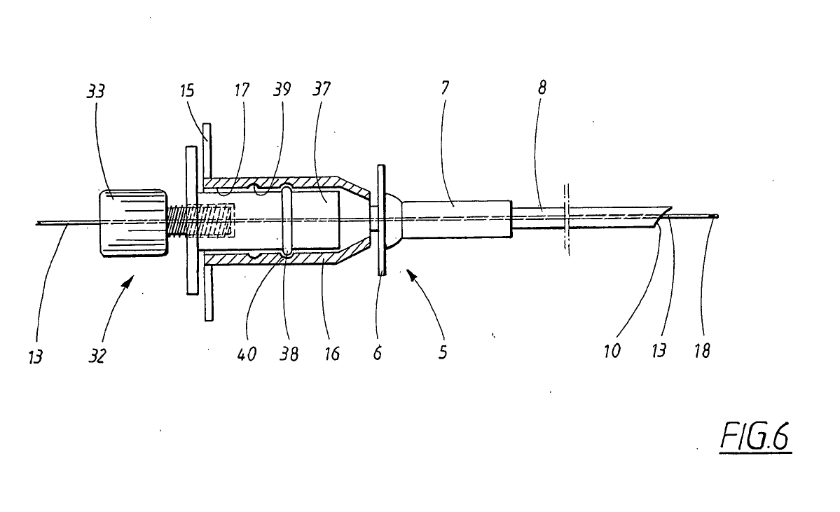

Furthermore, the cannula 8 is arranged to receive an optical fibre 13, which then runs through the cannula 8 in such a way that the end part 14 of the optical fibre 13 may assume a predetermined position in relation to the end part 10 of the cannula 8. For example, the optical fibre. 13 may be arranged in such a way that its end part 14 protrudes a small distance from the end part 10 of the cannula 8. The optical fibre 13 may also be brought to assume a protected position where its end part 14 is positioned a small distance inside the end part 10 of the cannula 8. The positioning of the optical fibre 13 will be described in detail below with reference to FIGS. 5 and 6. Suitably, the optical fibre 13 is dimensioned in such a way that it has a diameter that runs to 0.08-0.5 mm, but the invention is not limited to such dimensions, and the diameter might alternatively be of another magnitude.

The cannula 8 ends in a holding element 15, which suitably mainly is shaped as a plate or washer which comprises a cylindrical part 16 through which the cannula 8 extends and in which the cannula 8 is attached. In the holding element 15, a through hole 17 is defined (see FIG. 2) through which the optical fibre 13 runs. This hole 17 then also has its extension through the cylindrical part 16, such that the optical fibre 13 is allowed to run through the hole 17, the cylindrical part 16 and furthermore all the way to the point 10 of the cannula 8. The cannula 8 with its holding element 15 is then movable longitudinally in relation to the guide element 5, since the cannula 8 runs inside the hole 9 which in turn extends through the flange element 6 and the conducting tube 7.

When the measuring device 3 according to the invention shall be used, one holds the holding element 15 and presses the cannula 8 through the guide element 5, according to what is shown with arrows in FIG. 2 and FIG. 3, in what way the cannula 8 may assume the position that is shown in FIG. 1.

As mentioned above, the end part 14 of the optical fibre 13 is arranged to protrude a small distance out of the end part 10 of the cannula 8, accordingly out of that end of the cannula 8 that is intended to be positioned inside nucleus pulposus 2. As sensor element 18 is also arranged on the end part 14 of the optical fibre 13, which sensor element 18 according to the embodiment is constituted by a pressure sensor of the kind that is based on the use of a so-called Fabry-Pérot resonator. This type of pressure sensor is previously known, and is for example shown in the patent document EP 0639266.

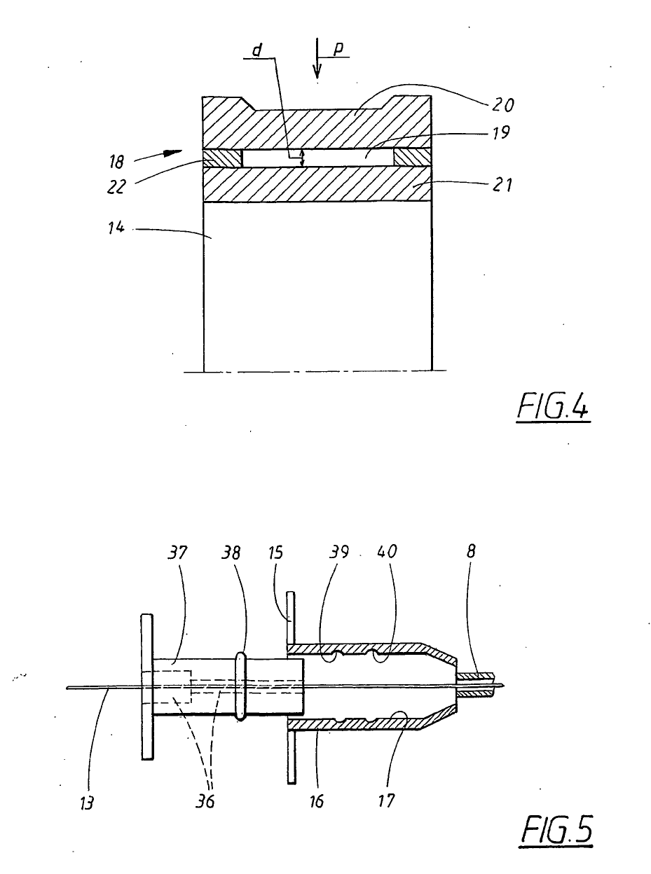

FIG. 4 is an enlarged cross-sectional view of the pressure sensor 18 that is used according to the present embodiment. The pressure sensor 18 is made around a cavity 19, i.e. a hollow space, which is defined by means of build-up of a first layer 20 and a second layer 21, which preferably are made of silicon. Between these layers, a layer is arranged which preferably is made of silicon dioxide 22, which suitably has a circular shape and which functions as a spacing element that defines a certain height d of the cavity 19, i.e. a certain distance between the first layer 20 and the second layer 21. The pressure sensor 18 is made by means of semiconductor technology and is attached against the end part 14 of the optical fibre. Thus a pressure sensor 18 is acquired by means of build-up of molecular layers, mainly silicon, alternatively silicon dioxide or a combination of silicon and silicon dioxide, and by means of an etching procedure. Suitably, a previously known bonding procedure is also used at the assembly of the different layers of the pressure sensor 18, according to what is described in said EP 0639266. Then a membrane 23 is formed above the cavity 19, where a mechanical bending of the membrane 23 may occur depending on the pressure p that surrounds the pressure sensor 18.

Regarding the dimensions of the pressure sensor 18, it preferably has a diameter that generally is of the same size as the optical fibre's 13 diameter, i.e. of the order of magnitude of one or some tenths of a millimetre. Furthermore, the pressure sensor 18 may in itself be used for measuring pressure within a wide range, generally up to roughly a pressure of 20 bar.

Hydrostatic pressure p that surrounds the sensor device 18 will thus affect this, and then in particular its membrane 23, such that the membrane 23 is bent mechanically and the dimensions of the cavity 19 are changed. If light is guided through the optical fibre 14 and into the cavity 19, the light will be reflected against the inner walls of the cavity 19 and give rise to a certain interference relation that depends on the degree of mechanical deflection of the membrane 23. This in itself known principle is used according to the invention for measuring the surrounding pressure p. For this purpose, the optical fibre 13 is connected to a measuring unit 24 during use, according to what is shown in FIG. 1. The measuring unit 24 comprises a light source in the form of a light-emitting diode 25, which is fed by means of a current source 26. Light from the light-emitting diode 25 is conducted to the optical fibre 13 via a first fibre branch 27 and a coupling 28.

The light from the light-emitting diode 25 is thus guided to the sensor device 18 and returned through the fibre 13 after having been reflected and modulated in the cavity 19. A certain part of the returned light radiation is guided via the coupling 28 to a photo sensitive element 29, for example a photo diode or a photo transistor, via a further fibre branch 30. The photo sensitive element 29 is in turn connected to an amplifying circuit 31 in which the received signal from the photo sensitive element 29 is transformed to an electrical output signal Uₒᵤₜ which constitutes a measure of the pressure p that surrounds the sensor device 18. By means of an intensity based measurement of the light that is reflected via the sensor device 18, a measurement of the pressure p is thus admitted.

Furthermore, the measuring device 3 according to the invention comprises a stopping device 32 (see FIG. 2 and FIG. 3) which is intended to be able to lock the optical fibre 13 in a certain fixed position in relation to the stopping device 32 itself. The stopping device 32 is furthermore in itself arranged to be locked to a certain position in relation to the holding element 15. For this purpose, the stopping device 32 comprises a stop screw 33 which is equipped with a through-hole 34 through which the optical fibre 13 may be brought. Furthermore, the stop screw 33 comprises a threaded part 35 which cooperates with the corresponding internally threaded hole 36 in a stop casing 37. This stop casing 37 may in turn be brought into the hole 17 in the holding element 15. For this purpose, the hole 17 has a slightly larger inner diameter than the outer diameter of stop casing 37.

Furthermore, the stop casing 37 is equipped with a bulge 38 that runs peripherally around the stop casing 37. The bulge 38 suitably consists of an O-ring which is known in itself, having an outer diameter that is slightly larger than the outer diameter of the rest of the stop casing 37. The outer diameter of the bulge 38 is also slightly larger than the outer diameter of the hole 17. The function of the stop casing 37 and its bulge 38 will now be described in detail.

In FIG. 5, a principle view is shown, partly in cross-section, of the stop casing 37 and the holding element 15 in a position before these parts have been assembled on their places during use. The figure also shows how the optical fibre 13 runs through these components and further through the cannula 8. For this purpose, the stop casing 37 is formed with an internally threaded hole 36 intended to receive the threaded part 35 of the stop screw 33, where the hole 36 has an extension through the whole stop casing 37. As mentioned above, the stopping device 32 is arranged for locking the optical fibre 13 in relation to the stop screw 33 (not apparent from FIG. 5). This suitably occurs when the stop screw 33 is screwed into the hole 36 of the stop casing 37.

The holding element 15 comprises a cylindrical part 16 through which an internal hole 17 runs. This hole 17 is equipped with at least one, but preferably two or more, peripheral recesses in the form of recessed grooves at predetermined axial positions. In FIG. 5, such a first groove 39 and a second groove 40 are shown. These grooves 39, 40 are formed in such a way that they can receive the bulge 38 of the stop casing 37, and they thus define predetermined positions for the positioning of the stop casing 37 in relation to the cylindrical casing 16. Since the optical fibre 13 may be fixed in relation to the stop screw 33, and thus also to the stop casing 37, the fibre 13 may also be brought to be positioned in one or more predetermined positions in relation to the cannula 8 during use.

In FIG. 6, a view of the stop casing 37 and the holding element 15 during use is shown, i.e. when the stop casing 37 has been positioned in a certain position in relation to the cylindrical part 16 and thus also in relation to the cannula 8. FIG. 6 also shows the guide element 5 and the cannula 8. According to the figure, the stop screw 33 is tightened, and thus the optical fibre 13 is fixed in relation to the stop screw 33 and also in relation to the stop casing 37. Furthermore, the stop casing 37 is inserted into the hole 17 of the holding element 15, such that the bulge 38 has snapped into a corresponding groove 40 in the hole 17. This means that the stop casing 37 is positioned in a fixed position in relation to the holding element 15.

It is furthermore shown in FIG. 6 how the cannula 8 runs through the flange element 6 and the conducting tube 7, and also how the optical fibre 13 extends a small distance in relation to the end part 10 of the cannula 8. The locking of the stop casing 37 in relation to the cylindrical part 16, by means of the bulge 38 that co-operates with the second groove 40, thus results in that the sensor device 18 on the end part of the fibre 13 will assume predetermined position in relation to the cannula 8. The included parts are suitably adapted in such a way that this predetermined position corresponds to that the sensor device 18 is just above the end part 10 of the cannula 8. This is then a position that is well suited for measuring the pressure p in nucleus pulposus.

If the stop casing 37 is brought backwards a small distance (i.e. to the left seen in FIG. 6), the bulge 38 will instead be able to be snapped into the first groove 39. This corresponds to a second predetermined position for the sensor device 18, which suitably is constituted by a position for the sensor device 18 just inside the end part 10 of the cannula 8, i.e. a protected position where the optical fibre 13 does not protrude from the cannula 8. An advantage of such an arrangement is that the sensor device 18 is protected against mechanical influence.

The protected position of the sensor device 18 may also be used for measuring the pressure p. This may be appropriate when one wishes to use the invention in such a way that pressure measurements are performed at two different defined positions of the sensor device 18, and then generates a total value concerning the pressure p based on both these pressure measurements. If one of the positions then is constituted by a protected position inside the end part 10 of the cannula 8, it is ensured that the measurement at this position only will relate to the hydrostatic pressure in nucleus pulposus, and no influence of possible mechanical pressure that possibly may act on the sensor device 18.

As an alternative to use a second position that is constituted by a protected position, the second position may be constituted by a further position outside the end part 10 of the cannula 8.

Furthermore, the cylindrical part 16 may in principle be formed with more than two recesses similar to those grooves 39, 40 that are shown in FIG. 6, in order to admit even more positioning possibilities for the optical fibre 13 in relation to the cannula 8.

According to what is apparent in FIG. 2 and FIG. 3, at least the front part of the cannula 8 is preferably equipped with a peripherally arranged part 41 with grooves or barb-like grooves. Such a part 41 may then contribute to an efficient fixation and a retaining effect of the cannula 8 when it is positioned inside nucleus pulposus. As an alternative to grooves or barbs, the part 41 may be formed with external threads which then have a corresponding retaining function.

In the following, the handling and function of the invention will be described. When a pressure measurement is to be performed with the measuring device 3 according to the invention, the skin of the human being 1 that the measurement is performed on is punctuated at a position close to nucleus pulposus 2. Then the conducting tube 7 is inserted into the hole that has been created. The tube 7 is then inserted so far that the flange element 6 is brought to bear on, and preferably also sewed to or in some other way fixed to, the skin of the human being 1.

The next step in the measurement method is to bring in the cannula 8 through the flange element 5 and the conducting tube 7. This may be facilitated by pressing a so-called faller stylet (not shown) into the cannula 8 and brings the latter to its position. Thus the end part 10 of the cannula 8 may be brought to the correct position for the current pressure measurement. When this has been done, the faller stylet is withdrawn.

The optical fibre 13 with its sensor arrangement 18 shall thereafter be brought into the cannula 8. Before this phase, the stopping device 32 has been mounted on the optical fibre 13. It is thus possible to adjust how far the fibre 13 shall protrude into the cannula 8 by means of the stop screw 33. When the stop screw 33 has been tightened into the cylindrical part 37 the optical fibre 13 has been fixed in relation to the stop screw 33. This corresponds to the position that is shown in FIG. 2 and FIG. 3.

The next step is that the stopping device 32 is pressed to be fixed against the holding element 15 of the cannula 8, i.e. the stop casing 37 is pressed into the cylindrical part 16 such that a certain fixed position is assumed. Then the sensor device 18 is fixed in a free determined position inside nucleus pulposus 2. This corresponds to the position that is shown in FIG. 6. If the stop casing 37 is equipped with several snap positions, for example two different grooves 39, 40 as shown in FIGS. 5 and 6, the optical fibre 13, and thus also the sensor device 18, may assume a corresponding number of alternative positions, according to the description above.

A basic principle behind the present invention is that the sensor device 18 for measuring the pressure p is arranged at the end part 13 of the optical fibre 13 and that the sensor device 18 during use is positioned in nucleus pulposus. An advantage with the invention is that an easily handled measuring device for efficient measurement of said pressure p is required. According to the preferred embodiment, the sensor device is constituted by a device for intensity based measurement of light that is reflected and modulated in a Fabry-Pérot resonator. A particular advantage with the invention is that it, according to the embodiment, comprises an articulated tube 7 that may be angled and aligned in a correct manner against nucleus pulposus, in order to guide the sensor device 18 against a correct measuring position. A further advantage is admitted by the invention if it, according to the embodiment, is formed with one or more snap positions, i.e. fixed positions for mounting of the stopping device 32 in relation to the holding element 15.

The invention is not limited to what is described above, but different embodiments are possible within the scope of the claims. The invention is for example not limited to measurements in nucleus pulposus, but may be used for measurements in other anatomic organs, for example the urinary bladder and prostate gland, where a signal related to hydrostatic pressure may be useful. The invention may also be used for measurements in tumours and certain muscles.

Furthermore, the invention may be used for measurements on both human beings and animals. The invention may also in principle be used at measurement of other physical magnitudes than the pressure p. For example, a sensor device for measurement of the temperature in an organ may be used. A sensor device of such a kind may then comprise a cavity containing a certain gas or a suitable solid compound that in turn expands when the temperature rises. By means of this expansion, a membrane may be affected in a way corresponding to the above. Such a temperature sensor may then also be mounted on the end part of an optical fibre similar to the one that has been described above.

1. Device for measuring a physical magnitude (p) in an anatomic organ in human beings and animals, comprising:
a tubular, hollow cannula arranged to be inserted into said human being or animal and comprising an end part that ends in said organ, and
an optical fibre adapted to be arranged with an extension inside said cannula,
a sensor device arranged on said optical fibre and during use is positioned in connection with said organ and is arranged for measuring said magnitude (p).

2. Device according to claim 1, further comprising a guide element arranged to be brought against the skin of said human being or animal, for guidance and fixation of said cannula.

3. Device according to claim 2, wherein said guide element comprises a flange element for bearing against the skin of said human being or animal, and a tubular element attached to said flange element and arranged for admitting insertion of said cannula.

4. Device according to claim 3, wherein said tubular element is attached in said flange element via a ball joint or a corresponding joint attachment.

5. Device according to claim 4, wherein said cannula is formed with an at least partly threaded or grooved peripheral surface.

6. Device according to claim 5, wherein said sensor device comprises a sensor for pressure (p), which sensor is arranged for measurements based on optical interference.

7. Device according to claim 6, wherein said sensor device is arranged on the end part of the optical fiber.

8. Device according to claim 7, comprising; a stopping device for insertion of said optical fiber and for locking the fiber in at least one predetermined position in relation to the cannula.

9. Device according to claim 8, wherein the stopping device comprises a part equipped with an external bulge which is arranged to co-operate with at least one correspondingly formed recess in a holding element arranged at the cannula.

10. Device according to claim 9, wherein said cannula is arranged for insertion into nucleus pulposus.

11. Method for measuring a physical magnitude (p) in an anatomic organ of human beings and animals, comprising:
insertion of a tubular, hollow cannula into said human being or animal, where the end part of the cannula is positioned in said organ; and
insertion of an optical fibre with an extension inside said cannula,
positioning of a sensor device arranged on said optical fibre in connection with said organ, and
measuring of said magnitude (p) by means of said sensor device.

12. Method according to claim 11, wherein said positioning of the sensor device comprises bringing a guide element against the skin of said human being or animal, and guiding and fixing said cannula during an insertion of said cannula through said guide element.

13. Method according to claim 11, wherein a measurement of pressure (p) based on optical interference.

14. Method according to claim 13, comprising locking said optical fibre in at least one predetermined position in relation to the cannula.

15. The device according to claim 1, wherein said cannula includes an at least partly threaded or grooved peripheral surface.

16. The device according to claim 1, wherein said sensor device comprises an optical pressure sensor.

17. The device according to claim 16, wherein said sensor is at the end part of the optical fiber.

18. The device according to claim 17, comprising a stopping device for insertion of said optical fiber and for locking the fiber in at least one predetermined position in relation to the cannula.

19. The device according to claim 18, wherein the stopping device comprises a part equipped with an external bulge which is arranged to co-operate with at least one correspondingly formed recess in a holding element arranged at the cannula.

20. The device according to claim 1, wherein said cannula for insertion into nucleus pulposus.
